(19) United States
(12) Patent Application Publication
(10) Pub. No.: US 2023/0405216 A1
(43) Pub. Date: Dec. 21, 2023

(54) Portable Carbon Dioxide Gas Injector

(71) Applicant: BEIJING ADVANCED MEDICAL TECHNOLOGIES, LTD. INC., Beijing (CN)

(72) Inventors: Hugh Qinghong Zhao, Pleasanton, CA (US); Alexander Nikanorov, Beijing (CN); Qinghua Zhao, Beijing (CN); Qing Liu, Beijing (CN); Helong Wang, Beijing (CN); Hong Du, Beijing (CN); Lele Lu, Beijing (CN)

(21) Appl. No.: 18/240,351

(22) Filed: Aug. 30, 2023

Related U.S. Application Data
(63) Continuation of application No. PCT/CN2021/093273, filed on May 12, 2021.

(30) Foreign Application Priority Data
Mar. 1, 2021 (CN) 202110223553.3

(51) Int. Cl. A61M 5/00

(52) U.S. Cl. CPC A61M 5/00; A61M 2005/006; A61M 2202/0225

(57) ABSTRACT

This application relates to the field of injection technology, especially a type of portable carbon dioxide gas injector. The technical solution of the invention includes a storage tube, one end of which is connected with a passage tube, and a passage valve is provided at the end of the passage tube away from the storage tube. A piston adapted to the injection device body is arranged inside the storage tube. A driving assembly for driving the piston is arranged on one side of the piston away from the passage tube, and a regulating assembly for adjusting the gas storage volume is arranged on the other side of the driving assembly away from the passage tube. The invention achieves quantitative control of the injection amount of carbon dioxide, which can alleviate the discomfort of patients.

### CROSS REFERENCE TO RELATED APPLICATIONS

The present application is a Continuation application of PCT Application No. PCT/CN2021/093273 filed on May 12, 2021, which claims the benefit of Chinese Patent Application No. 202110223553.3 filed on Mar. 1, 2021, the contents of which are incorporated herein by reference in their entirety.

### TECHNICAL FIELD

This application relates to the field of injection technology, especially a type of portable carbon dioxide gas injector.

### BACKGROUND TECHNOLOGY

As carbon dioxide can naturally exist in the human body, is biocompatible, soluble in blood, can be eliminated through exhalation from the lungs, and is non-toxic to the liver and kidneys, it is often used as a contrast agent for vascular angiography. In related techniques, when carbon dioxide needs to be injected into the body, the operator first uses a manual syringe to extract carbon dioxide from a cylinder assembly into the manual syringe, and then injects carbon dioxide into an external injection device that is in connection with the patient's body, and carbon dioxide enters the body through the external injection device to achieve vascular angiography.

Regarding the above related technical solutions, the inventor found that different blood vessels can withstand different volumes of carbon dioxide. Because the carbon dioxide inside the manual syringe is highly compressed, when injecting carbon dioxide into different parts of the body, the operator needs to exert a lot of force, which makes it difficult to accurately control the injection volume and may cause discomfort to the patient.

### Content of Invention

To be able to quantitatively control the injection volume of carbon dioxide and thus alleviate patient discomfort, the present application provides a portable carbon dioxide gas injector.

The portable carbon dioxide gas injector provided by the present application adopts the following technical solution:

The portable carbon dioxide gas injector includes a storage tube, one end of which is connected with a passage tube. The end of the passage tube away from the storage tube is connected with a passage valve. The storage tube is provided with a piston suitable for the injector and a driving assembly for driving the piston to move away from the passage tube, and a regulating assembly for regulating the gas storage volume. By using the above technical solution, when the patient needs to undergo angiography, the operator first connects the passage valve to the cylinder assembly, and carbon dioxide in the cylinder assembly enters the storage tube through the passage tube. When it is necessary to inject carbon dioxide into the patient's body, the driving assembly is used to drive the piston to inject carbon dioxide in the storage tube into the patient's body. When it is necessary to adjust the injection volume, the regulating assembly is used to adjust the gas storage volume in the storage tube, and then carbon dioxide in the storage tube is injected into the patient's body. The regulating assembly is able to regulate the gas storage volume in the storage tube, achieving precise quantitative control of the injection volume of carbon dioxide, thereby reducing patient discomfort. The piston separates the carbon dioxide from the driving assembly and the regulating assembly, reducing the possibility of other gases seeping into the carbon dioxide and ensuring the purity of the carbon dioxide. The passage valve separates the carbon dioxide from the outside world, reducing the possibility of other gases seeping into the carbon dioxide and further ensuring the purity of the carbon dioxide.

Preferably, the driving assembly includes a spring, one end of which is fixedly connected to the piston, and the other end of which is fixedly connected to the storage tube.

By using the above preferred technical solution, when the carbon dioxide in the cylinder assembly enters the storage tube through the passage valve and the passage tube, the spring is compressed by the carbon dioxide, and when it is necessary to inject carbon dioxide into the patient's body, the passage valve is switched to output state, and the spring is able to drive the piston towards the direction close to the passage tube, thereby achieving exhaust of the gas in the storage tube. The spring is set to be easy to operate, reducing the operator's effort in injecting carbon dioxide and lowering labor intensity, while increasing operational efficiency and convenience.

Preferably, the regulating assembly includes a regulating rod that passes through the storage tube and is threadedly connected to the storage tube. A gasket is fixedly mounted at one end of the regulating rod close to the piston.

By using the above preferred technical solution, when carbon dioxide gas enters the storage tube through the passage valve and the passage tube, it pushes the piston to move towards the direction away from the passage tube. When the end surface of the piston is close to the end surface of the regulating rod, injection can be stopped. When it is needed to inject different volumes of carbon dioxide into different parts of the body, the regulating rod can be rotated to adjust the position of the contacting surface of the regulating rod and the piston, thereby achieving adjustment of the gas storage volume in the storage tube. The regulating rod is set to be able to adjust the gas storage volume in the storage tube, making it easy to inject different volumes of carbon dioxide and reducing the possibility of discomfort to the patient. The gasket provides cushioning and shock absorption to the piston, reducing the possibility of injury to the piston caused by contact with the regulating rod during adjustment, and improving the safety of the piston.

The preferred embodiment includes the passage valve connected to the input assembly at the end away from the passage tube, and the end of the input assembly away from the storage tube is connected with the cylinder assembly.

In above configuration, the use of the input assembly enables smooth injection of carbon dioxide from the cylinder assembly into the storage tube when filling it, making the process more convenient.

Preferably, the cylinder assembly includes a connector that connects to the input assembly, and one end of the connector connects to the gas cylinder. A regulating knob is provided to adjust the gas pressure input into the storage tube through the connector.

The above configuration allows for easy injection of carbon dioxide into the input assembly from the gas cylinder by turning the regulating knob. The connector makes it easier to fill the input assembly with carbon dioxide from the gas cylinder, improving the convenience of use. The regulating knob allows the operator to control the flow of carbon dioxide into the storage tube, enhancing the convenience of operation. Also, the regulating knob can be used to adjust the pressure of carbon dioxide in the input assembly, increasing the versatility and convenience of use.

Preferably, the connector includes a pressure gauge to display the gas pressure inside the gas cylinder.

With above configuration, the gas pressure inside the gas cylinder can be observed by operators using the pressure gauge, allowing them to determine if a replacement cylinder is needed.

Preferably, the gas cylinder is detachably connected to the connector.

With above configuration, when the carbon dioxide gas in the gas cylinder is depleted, the detachable connection between the gas cylinder and connector facilitates easy replacement with a new cylinder, improving the operational life of the injector.

Preferably, a filter is provided on the input assembly to filter the gas passing into the storage tube.

With above configuration, the carbon dioxide gas passed through the filter is secondarily filtered, enhancing its purity and reducing the possibility of discomfort to patients.

Preferably, one end of the passage valve is connected to a Luer connector, and the other end of the Luer connector away from the passage valve is provided with a check valve.

With above configuration, when injecting carbon dioxide into the patient's body for angiographic imaging, the carbon dioxide gas passes through the passage valve, Luer connector and check valve, into the outer device connected to the patient's body, under the push of the spring and piston. The check valve ensures that carbon dioxide gas can only be discharged outwardly. The Luer connector is a non-leakage connector for trace fluids, reducing the possibility of external gases mixing with the carbon dioxide that is supposed to be injected into the patient's body, and the check valve further reduces the possibility of external gases entering the Luer connector and passage valve.

Preferably, the storage tube is covered with a protective sheath on its outer side to protect it against damage due to external impact.

With above configuration, the operator is able to have a non-slip grip on the storage tube during use. This enhances the safety of the storage tube and prevents damage due to external impact.

In summary, the present application provides the following technical effects:
1. By setting an adjusting assembly, the volume of gas that can be stored in the storage tube is adjusted through the adjusting assembly when needed. The adjusting assembly can adjust the gas storage volume of the storage tube, which achieves quantitative control of the injection volume of carbon dioxide, thereby alleviating discomfort in patients.
2. Setting a driving assembly facilitates the piston in the storage tube to expel the carbon dioxide to the external environment, making it unnecessary for the operator to exert great force during injection, reducing labor intensity, improving operation efficiency, and convenience.
3. Setting an adjusting knob facilitates the operator's control of the on-off of the carbon dioxide to be injected into the storage tube, improving operational convenience. Besides, the adjusting knob can adjust the gas pressure of the carbon dioxide to be injected into the input assembly, improving versatility and convenience of use.

### ATTACHED FIGURES

FIG. 1 is a structural schematic diagram of the injector in the embodiment one of the present application.

FIG. 2 is a structural schematic diagram of the embodiment one of the present application with protective cover of the injector hidden.

FIG. 3 is a sectional view highlighting the driving assembly and the adjusting assembly in the embodiment one of the present application.

FIG. 4 is a structural schematic diagram of the injector in the embodiment two of the present application.

FIG. 5 is a sectional view highlighting the driving assembly and the adjusting assembly in the embodiment two of the present application.

FIG. 6 is a partial sectional view highlighting the fixing component in the embodiment two of the present application.

FIG. 7 is an enlarged schematic diagram of location A in FIG. 6.

FIG. 8 is an angiographic effect diagram of carbon dioxide injected into the femoral artery of a human body.

FIG. 9 is an angiographic effect diagram of carbon dioxide injected into the aorta of a human body.

In the figures, 01 refers to the storage tube; 011 refers to the piston; 012 refers to the sealing ring; 013 refers to the scale line; 02 refers to the passage tube; 03 refers to the passage valve; 031 refers to the rotary handle; 04 refers to the input assembly; 041 refers to the first connecting tube; 042 refers to the second connecting tube; 05 refers to the cylinder assembly; 051 refers to the connector; 0511 refers to the pressure gauge; 052 refers to the gas cylinder; 053 refers to the adjusting knob; 06 refers to the driving assembly; 061 refers to the spring; 07 refers to the adjusting assembly; 071 refers to the adjusting rod; 072 refers to the adjusting cap; 073 refers to the gasket; 074 refers to the moving plate; 075 refers to the docking block; 076 refers to the moving rod; 0761 refers to the fixing hole; 077 refers to the handle; 08 refers to the filter; 09 refers to the Luer connector; 10 refers to the check valve; 11 refers to the protective cover; 111 refers to the observation groove; 12 refers to the fixing component; 121 refers to the fixing rod; 122 refers to the fixing box; 123 refers to the moving block; 124 refers to the limiting spring; and 125 refers to the pull ring.

### DETAILED DESCRIPTION OF EMBODIMENTS

This application is further explained in detail in conjunction with the following attached drawings.

### Embodiment 1

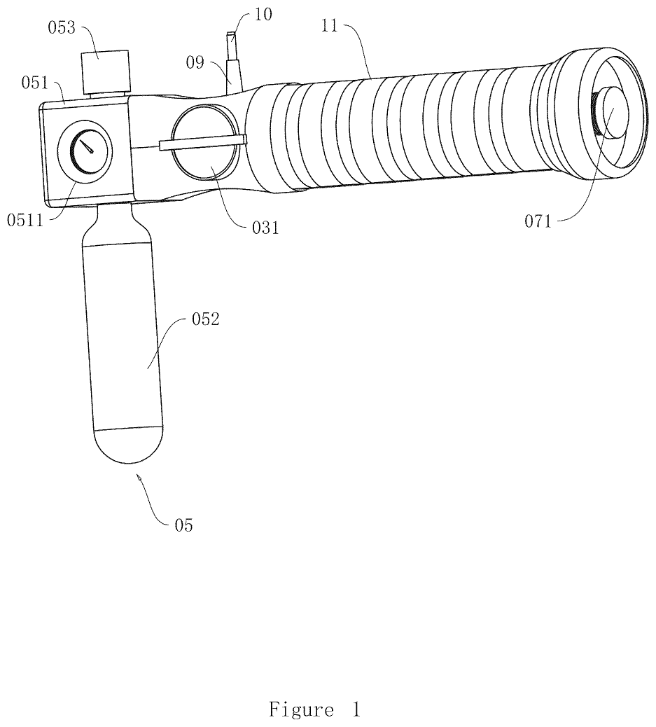

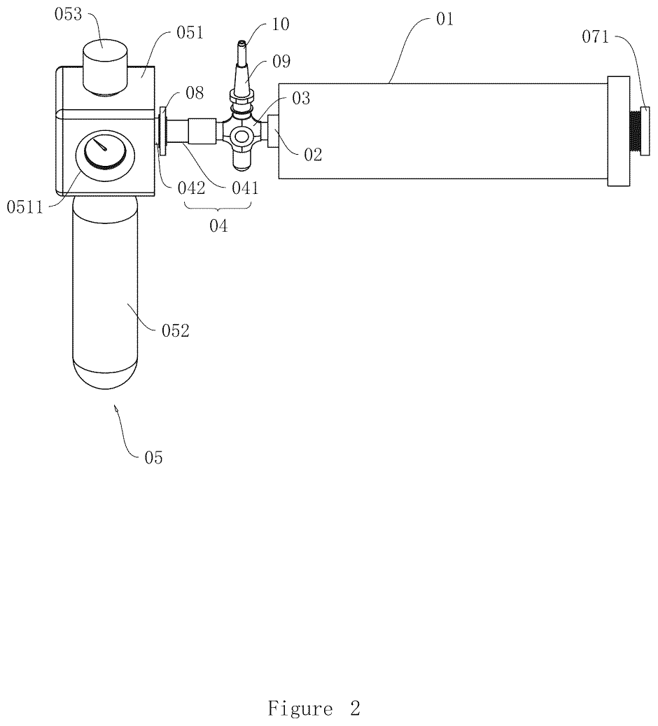

Referring to FIGS. 1 and 2, the present application provides a portable carbon dioxide gas injector, comprising a horizontally arranged storage tube 01. The storage tube 01 is approximately a stepped cylinder. One end of the storage tube 01 is connected to a coaxial passage tube 02, and the other end of the passage tube 02, away from the storage tube 01, is equipped with a passage valve 03, which is a three-way valve. A rotary handle 031 is fixed on the passage valve 03. The end of the passage valve 03 away from the storage tube 01 is connected to an input assembly 04, and a cylinder assembly 05 is arranged at the end of the input assembly 04 away from the passage valve 03. External injection devices connected to blood vessels of the patient are arranged on one side of the passage valve 03.

When the patient needs to undergo angiography, the operator first switches the passage valve 03 to make the input assembly 04 in communication with the passage tube 02. Then, the cylinder assembly 05 and the input assembly 04 are used to input carbon dioxide into the storage tube 01. After the input is completed, the passage valve 03 is switched to make the passage tube 02 in communication with the external injection devices. The carbon dioxide in the storage tube 01 is injected into the patient's body, allowing the patient to undergo angiography. The setting of the passage valve 03 integrates the cylinder assembly 05, the storage tube 01, and the external injection device, reducing the possibility of external gas permeating into the carbon dioxide gas, ensuring the purity of the carbon dioxide, and reducing the possibility of discomfort in patients caused by other gases mixed with the carbon dioxide. The setting of the passage valve 03 makes it easy to inject carbon dioxide into the patient's body by only switching the passage valve 03, improving the ease of use. The rotary handle 031 can only be operated in two states, which are making the input assembly 04 and the passage tube 02 in communication and making the passage tube 02 and the external injection devices in communication. This prevents incorrect operation by the operator, making the switching of the rotary handle 031 a foolproof operation.

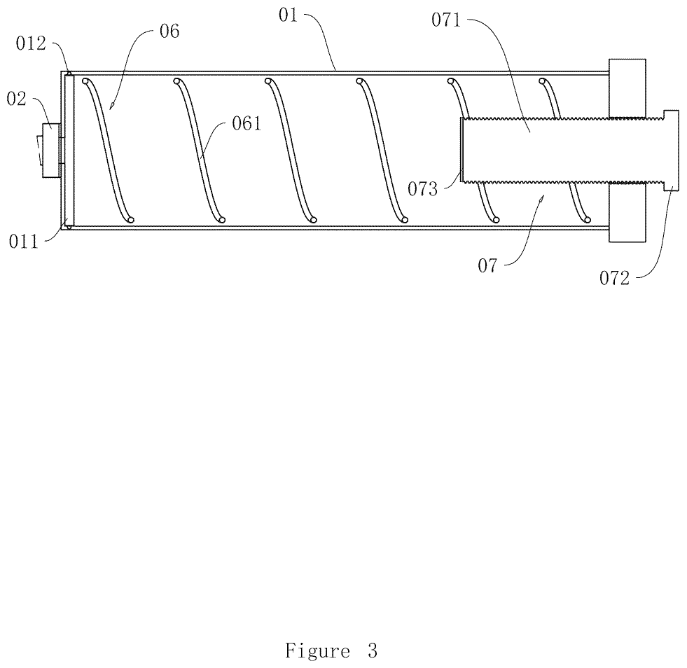

Referring to FIG. 3, a piston 011, coaxial with the storage tube 01, is arranged inside the storage tube 01. The piston 011 is compatible with the storage tube 01. A sealing ring 012 is set on the periphery of the piston 011. On the side of the piston 011 away from the passage tube 02, a driving assembly 06 is provided to drive the piston 011 to move. The driving assembly 06 uses a spring 061 that is coaxial with the storage tube 01. One end of the spring 061 is fixedly connected to the end face of the piston 011 away from the passage tube 02, and the other end of the spring 061 is fixedly connected to the inner wall of the storage tube 01.

When injecting carbon dioxide into the patient's body, the passage valve 03 is switched to allow the carbon dioxide in the cylinder assembly 05 to enter the storage tube 01. As the carbon dioxide inside the storage tube 01 is highly compressed gas, the piston 011 and the spring 061 move towards the end away from the passage tube 02 under the pressure of the carbon dioxide. After a certain amount of carbon dioxide is injected into the storage tube 01, the passage valve 03 is switched to make the passage tube 02 in communication with the external injection devices. The carbon dioxide is pushed into the external injection devices by the spring 061. The piston 011 separates the carbon dioxide from the driving assembly 06 and adjusting assembly 07, reducing the possibility of other gases permeating into the carbon dioxide gas, ensuring the purity of the carbon dioxide. The sealing ring 012 further improves the sealing of the space storing the carbon dioxide gas. The spring 061 facilitates the discharge of carbon dioxide from the storage tube 01, eliminating the need for the operator to exert much force to inject carbon dioxide into the patient's body, reducing labor intensity, and improving operational efficiency and convenience.

Referring to FIG. 3, the adjusting assembly 07 is provided on the side of the piston 011 away from the passage tube 02 to adjust the gas storage volume. The adjusting assembly 07 includes an adjusting rod 071 coaxial with the storage tube 01. The adjusting rod 071 is threadedly connected to the storage tube 01, and there is a threaded hole on the storage tube 01 for the adjusting rod 071 to rotate. The end of the adjusting rod 071 away from the piston 011 is fixedly connected to an adjusting cap 072, and the other end of the adjusting rod 071, near the piston 011, is fixedly connected to a gasket 073.

When the carbon dioxide enters the storage tube 01 through the passage valve 03 and the passage tube 02, the carbon dioxide pushes the piston 011 to move towards the end away from the passage tube 02. The piston moves until the end face of the piston 011 is in contact with the end face of the adjusting rod 071. When the gas storage volume inside the storage tube 01 needs to be adjusted, the operator rotates the adjusting rod 071 along the axis of the storage tube 01 to change the position of the contact surface of the piston 011 and the adjusting rod 071 to realize the adjustment of gas storage volume in storage tube 01. The adjusting rod 071 realizes the adjustment of the gas storage volume inside the storage tube 01, facilitating the injection of carbon dioxide of different volumes into the human body. This reduces the possibility of discomfort in patients caused by injecting too much carbon dioxide. The threaded connection between the adjusting rod 071 and the storage tube 01 reduces the possibility of the adjusting rod 071 being pushed by highly compressed carbon dioxide, which facilitates the quantitative control of the injection of carbon dioxide. The adjusting cap 072 facilitates the rotation of the adjusting rod 071, improving ease of use. The gasket 073 provides damping and slows down the movement of the piston 011 when it contacts the adjusting rod 071, reducing the possibility of the piston 011 being damaged during rapid movements, improving the safety of the piston 011, and extending its service life.

Referring to FIG. 2, the cylinder assembly 05 includes a connector 051 connected to the input assembly 04. One side of the connector 051 is connected to a gas cylinder 052, which stores highly compressed carbon dioxide. The gas cylinder 052 is detachably connected to the connector 051. The connector 051 has an adjusting knob 053 for regulating the gas pressure input into the storage tube 01. The connector 051 is provided with a pressure gauge 0511 for displaying the gas pressure inside the gas cylinder 052.

When inputting carbon dioxide into the storage tube 01, the adjusting knob 053 is rotated to allow carbon dioxide from the gas cylinder 052 to be input into the storage tube 01 through the connector 051. The connector 051 facilitates the input of carbon dioxide into the storage tube 01, and the adjusting knob 053 allows the operator to control the pressure of carbon dioxide injected into the storage tube 01, making the use of the injector more versatile and convenient. The pressure gauge 0511 allows the operator to observe the remaining gas pressure in the gas cylinder 052 and decide whether to replace the gas cylinder 052. With the detachable connection between the gas cylinder 052 and the connector 051, the operator can easily replace the gas cylinder 052, making the use of the system more convenient.

Referring to FIG. 2, the input assembly 04 includes a first connecting tube 041 and a second connecting tube 042 coaxial with the first connecting tube 041. A filter 08 coaxial with the first connecting tube 041 is fixed on an end surface near the second connecting tube 042. The filter 08 is designed to perform secondary cleaning by filtration on the carbon dioxide charged into the storage tube 01, further improving the purity of the carbon dioxide and reducing the likelihood of patients experiencing discomfort symptoms.

Referring to FIGS. 2 and 3, one end of the passage valve 03 is connected to a Luer connector 09, of which the end away from the passage valve 03 is connected to a check valve 10. When injecting carbon dioxide into the patient's body, the passage valve 03 is rotated to connect the passage tube 02 to the external injection device. Then, under the action of the spring 061 and the piston 011, the carbon dioxide passes through the passage valve 03, Luer connector 09, and check valve 10 to enter the external injection device connected to the patient's body, allowing blood vessels to perform angiographic imaging. The Luer connector 09 reduces the likelihood of outside gas infiltrating into the carbon dioxide injected into the patient's body, ensuring the purity of carbon dioxide and reducing patient discomfort. The check valve 10 only allows the carbon dioxide to discharge outwardly, further reducing the possibility of outside gas entering the passage valve 03.

Referring to FIGS. 1 and 2, a protective cover 11 is provided on the storage tube 01, cylinder assembly 05, and input assembly 04 for protection. The protective cover 11 facilitates the operator's grip on the storage tube 01, and protects the storage tube 01, cylinder assembly 05, and input assembly 04 from being damaged by external impact, thereby improving their safety.

In summary, the process of this application is as follows: when carbon dioxide needs to be injected into the patient's body, the handling knob 031 is rotated to connect the input assembly 04 with the passage tube 02. Then, the knob 053 is rotated to input the carbon dioxide into the storage tube 01 from the gas cylinder 052, and the spring 061 is compressed by the highly compressed carbon dioxide gas. After the inputting process is completed, when carbon dioxide needs to be injected into the patient's body, the handling knob 031 is rotated to connect the passage tube 02 with the external injection device, and the spring 061 forces the carbon dioxide in the storage tube 01 to inject into the patient's body for angiographic imaging of blood vessels. When adjusting the gas storage volume in the storage tube 01, the adjusting rod 071 is rotated to move along the axis of the storage tube 01, so that the position where the piston 011 and the adjusting rod 071 meets is changed to adjust the gas storage volume. When the carbon dioxide in the gas cylinder 052 is insufficient, the gas cylinder 052 can be detached from the connector 051 and replaced with a new gas cylinder 052.

### Embodiment 2

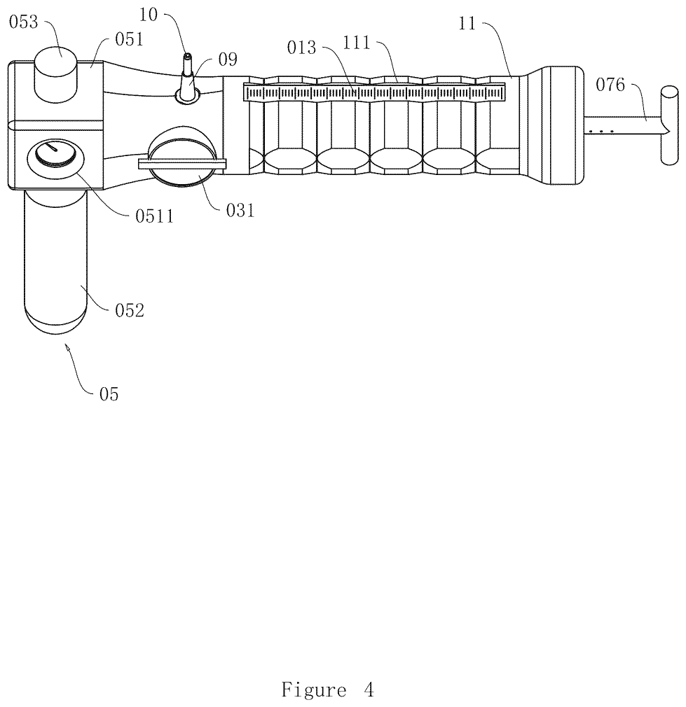

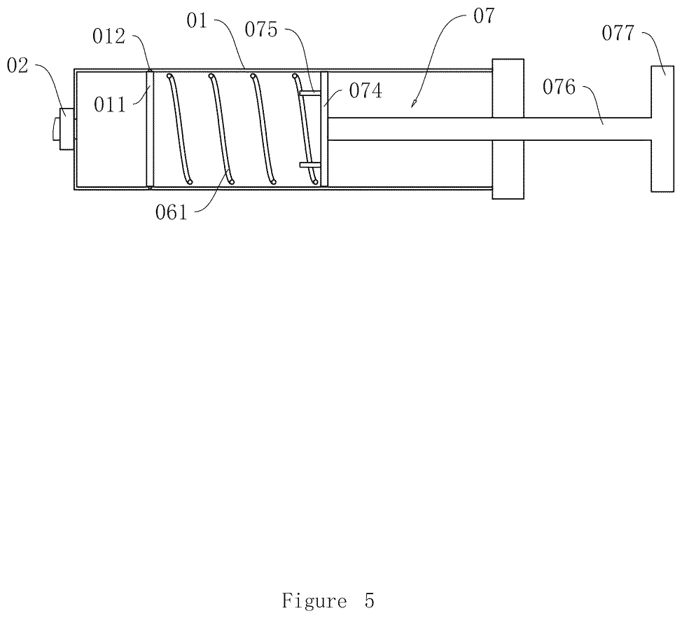

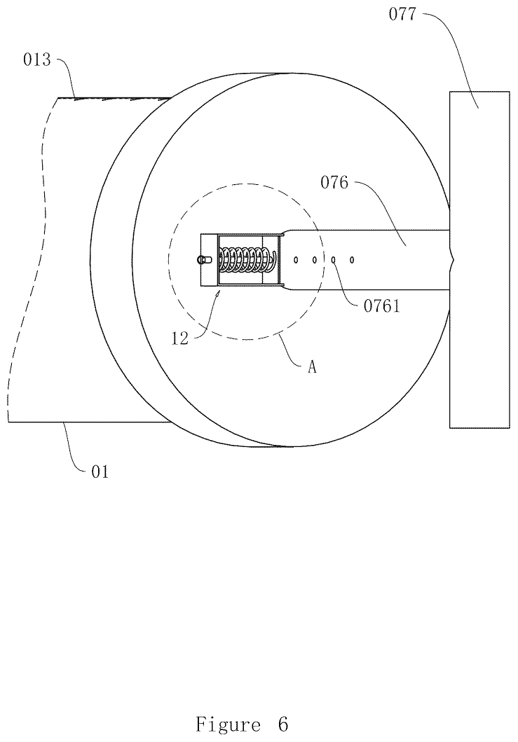

Referring to FIGS. 4 and 5, the difference between Embodiment 2 and Embodiment 1 is that the adjusting assembly 07 of embodiment 2 includes a moving plate 074 coaxial with the storage tube 01, adapted to the storage tube 01, located at the end of the piston 011 away from the passage tube 02, with one end of the spring 061 fixedly connected to the piston 011 and the other end fixedly connected to the moving plate 074. Two horizontal docking blocks 075 are fixed on the end face of the moving plate 074 close to the piston 011, symmetrically arranged along the axis of the moving plate 074. A moving rod 076 coaxial with the storage tube 01 is fixed on the end of the moving plate 074 away from the piston 011, and a handle 077 is fixed on the end of the moving rod 076 away from the moving plate 074. Referring to FIG. 6, a fixing component 12 is provided on the storage tube 01 for fixing the moving rod 076, and a through hole is provided on the storage tube 01 for the moving rod 076 to move. Scale lines 013 are provided on the side surface of the storage tube 01, and an observation groove 111 is provided on the protective cover 11 for the operator to observe the scale lines 013.

When quantitatively injecting carbon dioxide into the patient's body, the moving rod 076 is fixed by using the fixing component 12. As the length of the docking block 075 and the moving rod 076 are determined, after filling the storage tube 01 with highly compressed carbon dioxide, the piston 011 abuts against the docking block 075 to define the amount of carbon dioxide in the storage tube 01 quantitatively. The operator can observe the scale lines 013 corresponding to the piston 011 to determine the gas storage capacity of the storage tube 01, achieving accurate control over the injection amount of carbon dioxide to relieve patient discomfort. The docking block 075 facilitates the quantitative control of carbon dioxide injection amount, improving the convenience of use; the moving rod 076 is combined with the fixing component 12 to adjust the gas storage volume in the storage tube 01, making it easier for storage tube 01 to accommodate and output different volumes of carbon dioxide to reduce the likelihood of patient discomfort caused by overdoses; the fixing component 12 reduces the possibility of the adjustment rod 071 being pushed due to the high compression of carbon dioxide, further facilitating the quantification control of the carbon dioxide injection amount; the handle 077 facilitates the movement of the moving rod 076, improving the convenience of operation; the scale lines 013 enable the operator to observe the gas storage capacity of the storage tube 01, improving usability, and the observation groove 111 can be used to observe the scale lines 013.

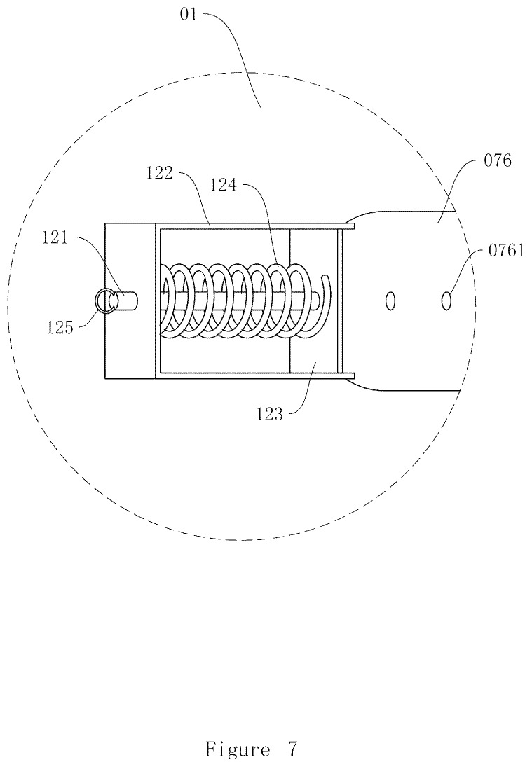

Referring to FIGS. 6 and 7, the fixing component 12 includes a horizontally set fixing rod 121, with its axis perpendicular to the axis of the moving rod 076. Multiple fixing holes 0761 are uniformly provided along the extension direction of the moving rod 076 for the insertion of the fixing rod 121. The outer side of the fixing rod 121 is equipped with a hollow fixing box 122, which is fixedly connected to the storage tube 01. The fixing rod 121 passes through the box wall of the fixing box 122 near and away from the moving rod 076, and the fixing box 122 has a matching moving block 123 inside, which is slidably connected to the fixing box 122. The fixing rod 121 passes through the moving block 123 and is fixedly connected to it, and there is a through-hole on the moving block 123 for inserting the fixing rod 121.

To achieve quantitative adjustment of the gas storage volume inside the storage tube 01, firstly move the moving rod 076 by a certain distance, then insert the fixing rod 121 into the corresponding fixing hole 0761, next input highly compressed carbon dioxide into the storage tube 01 to make the piston 011 abut against the docking block 075 and finally store a quantitative amount of gas in the storage tube 01. The fixing rod 121 can fix the moving rod 076, reducing the possibility of the moving rod 076 being moved by the impact of highly compressed carbon dioxide and improving the convenience of use. The fixing box 122, which cooperates with the fixing block, can fix the fixing rod 121, enabling it to fix the moving rod 076. The multiple fixing holes 0761 facilitate the fixing of the fixing rod 121 to different positions of the moving rod 076, making it easy to adjust the gas storage volume inside the storage tube 01 and improving the versatility and convenience of use.

Referring to FIGS. 6 and 7, a limiting spring 124 is sleeved on the fixing rod 121. One end of the limiting spring 124 is fixedly connected to the inner wall of the fixing box 122 away from the moving rod 076, and the other end is fixedly connected to the moving block 123. A pull ring 125 is fixed at one end of the fixing rod 121 away from the moving rod 076.

To fix the moving rod 076 in different positions, firstly move the fixing rod 121 away from the moving rod 076 by pulling the pull ring 125, compressing the limiting spring 124 with the action of the moving block 123, then move the moving rod 076 along the extension direction of the storage tube 01 to the desired position, and finally release the pull ring 125 to enable the limiting spring 124 to drive the fixing rod 121 into the corresponding fixing hole 0761 for fixing the moving rod 076. The limiting spring 124 can facilitate automatic reset of the moving rod 076, and when the fixing rod 121 is inserted into the fixing hole 0761, the limiting spring 124 can limit the fixing rod 121, reducing the possibility of it being ejected outward due to the impact of carbon dioxide, and improving the stability of fixing the moving rod 076. The pull ring 125 facilitates the movement of the fixing rod 121 by the operator, improving the convenience of use.

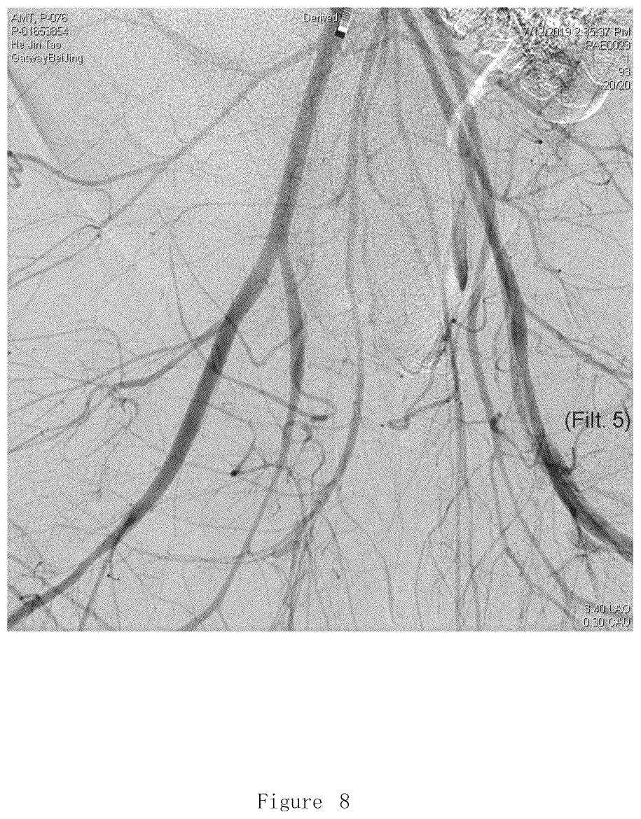

Referring to FIG. 8, when the femoral artery of a human body needs to be radiographed, carbon dioxide is injected into the body, and after injection, the femoral artery will be shown in the corresponding angiographic image.

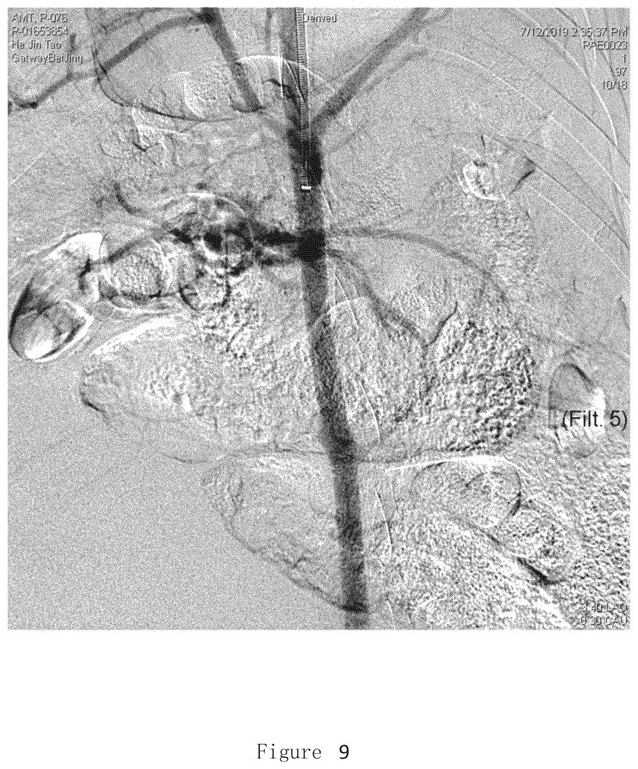

Referring to FIG. 9, when the aorta of a human body needs to be radiographed, carbon dioxide is injected into the body, and after injection, the aorta will be shown in the corresponding angiographic image.

This particular embodiment is only an explanation of this application and is not a limitation of it. Those skilled in the art can make non-inventive modifications to this embodiment based on the description herein, but all fall within the scope of protection of the patent law as long as they are within the range defined by the claims of this application.

What is claimed is:

1. The portable carbon dioxide gas injector is characterized by including a storage tube (01), one end of which is connected with a passage tube (02), and a passage valve (03) is provided at the end of the passage tube (02) away from the storage tube (01). A piston (011) adapted to the storage tube (01) is arranged inside the storage tube (01). A driving assembly (06) for driving the piston (011) is arranged on one side of the piston (011) away from the passage tube (02), and a regulating assembly (07) for adjusting the gas storage volume is arranged on the other side of the driving assembly (06) away from the passage tube (02).

2. The portable carbon dioxide gas injector according to claim 1, has the following characteristics: a driving assembly (06) that comprises a spring (061), one end of which is fixedly connected with the piston (011), and the other end of the spring (061) is fixedly connected with the storage tube (01).

3. The portable carbon dioxide gas injector according to claim 2, has the following characteristics: a regulating assembly (07) that comprises an adjusting rod (071) passing through the storage tube (01), and the adjusting rod (071) is threaded with the storage tube (01). A gasket (073) is fixedly arranged at one end of the adjusting rod (071) close to the piston (011).

4. The portable carbon dioxide gas injector according to claim 1, has the following characteristics: the end of the passage valve (03) away from the passage tube is connected with the input assembly (04), and a cylinder assembly (05) is provided at the end of the input assembly (04) away from the storage tube (01).

5. The portable carbon dioxide gas injector according to claim 4, has the following characteristics: the cylinder assembly (05) comprises a connector (051) connected with the input assembly (04), and one side of the connector (051) is connected with a gas cylinder (052). An adjusting knob (053) for adjusting the gas pressure inside the storage tube (01) is provided on the connector (051).

6. The portable carbon dioxide gas injector according to claim 5, has the following characteristics: a pressure gauge (0511) for displaying the gas pressure inside the gas cylinder (052) is provided on the connector (051).

7. The portable carbon dioxide gas injector according to claim 5, has the following characteristics: the gas cylinder (052) is detachably connected with the connector (051).

8. The portable carbon dioxide gas injector according to claim 4, has the following characteristics: a filter (08) for filtering the gas injected into the storage tube (01) is provided on the input assembly (04).

9. The portable carbon dioxide gas injector according to claim 1, has the following characteristics: one end of the passage valve (03) is connected with a Luer connector (09), and a one-way valve (10) is provided at the end of the Luer connector (09) away from the passage valve (03).

10. The portable carbon dioxide gas injector according to claim 1, has the following characteristics: a protective case (11) for protecting the storage tube (01) is arranged outside the storage tube (01).
